(19) United States
(12) Patent Application Publication
(10) Pub. No.: US 2009/0232790 A1
(43) Pub. Date: Sep. 17, 2009

(54) Kit of Lyophilized Thrombin and Lyophilized Fibrinogen Used to Compound Fibrin Membrane, and Its Application

(76) Inventor: Kieu Hoang, Agoura Hills, CA (US)

Correspondence Address: MEREK, BLACKMON & VOORHEES, LLC, 673 S. WASHINGTON ST, ALEXANDRIA, VA, 22314, US

(21) Appl. No.: 11/990,203

(22) PCT Filed: Jul. 11, 2006
(86) PCT No.: PCT/US2006/026739

§ 371 Date: Jul. 15, 2008

(30) Foreign Application Priority Data
Aug. 8, 2005 (CN) 200510028577.4

(51) Int. Cl. A61K 38/48

(52) U.S. Cl. 424/94.64

(57) ABSTRACT

A kit of lyophilized thrombin and lyophilized fibrinogen comprises fibrinogen of 50-100 mg/ml, thrombin of 100-1000 IU/ml and 20-60 mmol/L CaCl₂ used to compound fibrin membrane. The lyophilized thrombin and lyophilized fibrinogen are applied in a clinical tumor operation for reducing the risk of tumor metastasis after the operation. The solutions of thrombin and fibrinogen are daubed on the surface of a tumor, where they mix and form a solid-meshy fibrin membrane that prevents pervasion of tumor cells caused by incision and trauma in the tumor operation, thereby reducing the risk of palindromia and metabasis after the operation and improving the patient's life span.

### BACKGROUND OF THE INVENTION

1. Field of the Invention

The present invention relates to a kit of lyophilized thrombin and lyophilized fibrinogen and its usage to prevent tumor cell pervasion caused by incision and trauma in tumor operations.

In tumor operations, incision and trauma usually cause tumor cell pervasion, which can increase the risk of tumor palindromia and metabasis after tumor operations, and shorten the life span of the patients.

2. The Prior Art

A compound of fibrinogen and thrombin has been used to reduce lumps after radical mamma and lump exsection. More specifically, there is a tendency for lumps to develop where the mamma or lump has been removed, and the compound of fibrinogen and fibrin has been daubed in these places and reduced the number and/or size of lumps that tend to form there. The compound has also been used as a topical hemostasis drug in the treatment of the surface of burns, abdominal incisions of general surgery, oozing of blood in liver operations and blood vessel surgery.

3. SUMMARY OF THE INVENTION

In the present invention, a compound of fibrinogen and thrombin is used to prevent dissociative tumor cell pervasion.

A kit of the present invention produces a solid-meshy fibrin membrane to prevent dissociative tumor pervasion.

It is an object of the present invention to provide a kit for compounding fibrin membrane.

Another object of the present invention is the application of the kit, which includes lyophilized thrombin and lyophilized fibrinogen, as well as water and a solvent, in a tumor operation. The kit further includes instructions for the use of the kit, which is commonly called the kit instruction manual. The lyophilized thrombin and lyophilized fibrinogen are used to compound fibrin membrane, wherein the kit comprises fibrinogen of 50-100 mg/ml, for which water can be used as the solvent, thrombin of 100-1000 IU/ml, and 20-60 mmol/L CaCl₂ as the solvent for the thrombin. The thrombin and fibrinogen are sourced from human blood. The kit is applied to prevent pervasion of tumor cells caused by incision and trauma in a tumor operation. The fibrin membrane can reduce the risk of palindromia and metabasis of the tumor after treatment and improve the life span of patients. The fibrin membrane has a good biological compatibility and convenient usage.

### BRIEF DESCRIPTION OF THE DRAWINGS

Other objects and features of the present invention will become apparent from the following detailed description considered in connection with the accompanying drawings. It should be understood, however, that the drawings are designed for the purpose of illustration only and not as a definition of the limits of the invention.

FIG. 1 and FIG. 2 are electron micrographs of fibrin membrane compounded by daubing a solution of lyophilized thrombin and a solution of lyophilized fibrinogen one layer after another on a glass slide.

FIG. 3A1 and FIG. 3A2 are electron micrographs of a control without fibrin membrane.

FIG. 3B1 and FIG. 3B2 are electron micrographs of a material surface with a fibrin membrane according to the present invention in a tumor cell pervasion experiment.

FIG. 3C1 and FIG. 3C2 are electron micrographs of fibrin membrane in a tumor cell pervasion experiment involving a fibrin membrane treatment according to the present invention.

### DETAILED DESCRIPTION OF THE PREFERRED EMBODIMENTS

The following examples serve to further illustrate the invention.

The effects of fibrinogen and thrombin on the process of thrombosis are well known. There are many products from fibrinogen and thrombin, but the products are used to stanch after operations in most cases. According to the present invention, a solid-meshy fibrin membrane compounded by fibrinogen and thrombin is used to prevent the pervasion of tumor cells. By the present invention, a kit of fibrinogen and thrombin for fibrin membrane is provided. The source of the fibrinogen and thrombin is human blood, the concentration of fibrinogen is 50-100 mg/ml, the concentration of thrombin is 100-1000 IU/ml, and the concentration of CaCl₂ is 20-60 mmol/L. In a preferred embodiment, the kit comprises a bottle of 100-200 mg lyophilized fibrinogen with a solvent of 2 ml water for injection [(WFI)] and a bottle of 200-2000 IU lyophilized thrombin with a solvent of 2 ml 20-60 mmol/L CaCl₂ solution. In a more preferred embodiment, the concentration of fibrinogen is 60-80 mg/ml, the concentration of thrombin is 300-800 IU/ml, and the concentration of CaCl₂ is 30-50 mmol/L CaCl₂. In a most preferred embodiment, the concentration of fibrinogen is 70 mg/ml, the concentration of thrombin is 400-600 IU/ml, and the concentration of CaCl₂ is 40 mmol/L CaCl₂. The kit further includes the kit instruction manual. The present invention is also directed to a method of using the kit.

The fibrinogen and thrombin sourced from human blood are lyophilized and put into separate solutions. In experiments using the present invention, a thin and smooth layer of fibrinogen solution is daubed on the surface of a glass slide, or on the bottom surfaces of cell culture inserts in an assay kit, to form a coating. After about 5 seconds, a thin and smooth layer of thrombin solution is daubed sequentially on the fibrinogen coating. The daubing of the fibrinogen solution and then the thrombin solution is repeated about 3-5 times. A solid-meshy fibrin membrane forms quickly. The diameter of the fibrin membrane mesh is less than 0.6 μm in its biggest dimension and far smaller than human tumor cells of 10-100 μm. The fibrin membrane can hold back the human tumor cells and prevent pervasion.

Thus, the fibrinogen and thrombin solutions are daubed alternately with one another on the local surface of tumor tissue, one layer at a time. The solid-meshy fibrin membrane that forms on the local tissue surface prevents dissociative tumor cell pervasion in operations, reduces the risk of palindromia and metabasis of tumors after treatment, and improves the life span of patients. The fibrin membrane has a good biological compatibility and convenient usage.

### EXAMPLE 1

### Generation of Fibrin Membrane

(1) The fibrinogen solution and the thrombin solution of the kit according to the present invention were each daubed three times, alternately, one layer at a time, on a 0.8 cm×0.8 cm slide surface (compounded by 450 IU/ml thrombin and 40 mmol/L CaCl₂).

(2) The layers of the fibrinogen solution and thrombin solution were air dried and inspected with an electron microscope.

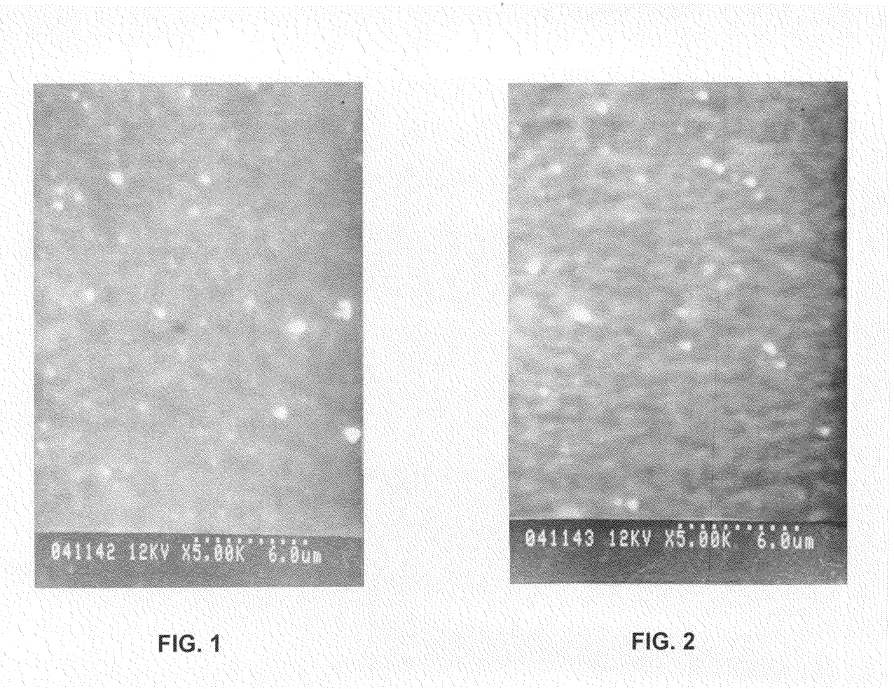

FIG. 1 and FIG. 2 are electron microscope photographs of the dried fibrinogen and thrombin layers. The amplification ratio is 5000. From these photographs, the smooth fibrin membrane surface can be seen. There are no distinct holes in the FS fibrin membrane. It can be induced from the amplified scale that the mesh bore diameter is less than 0.6 μm. Human tumor cell size is 10-100 μm. Thus, the fibrin membrane compounded by the human blood fibrinogen and thrombin can hold back human tumor cells and prevent pervasion.

### EXAMPLE 2

From a fibrin membrane kit according to the present invention for preventing dissociative tumor cell pervasion:

(1) A thin and smooth fibrin membrane was produced on the bottom surfaces of the cell culture inserts that are placed into the wells of the tissue culture plate of a Cell Invasion Assay Kit ECM550 commercially available from Chemicon International of Temecula, Calif. to form a coating thereon by using the method of EXAMPLE 1. Then, a cell invasion experiment was carried out according to the instructions provided in the cell invasion assay kit. The invasive cells in the cell suspensions that were placed in the inserts included carcinoma ventriculi cell lines (tumor of stomach), human gastric adenocarcinoma cell line KN45, and AGS human cultured gastric adenocarcinoma cells from Ruijing Hospital of Shanghai, China, as well as human breast cancer cells MDA-MB-231 and colon cancer cells Ls174T from SBI (System Biosciences of Mountain View, Calif.).

(2) In accordance with the instructions in the cell invasion assay kit, the spent medium was discarded, the inner membrane was cleared using a cotton-tipped swab, and the inserts were stained for 20 minutes and air dried. There was no fibrinogen or thrombin in the control wells of the tissue culture plate.

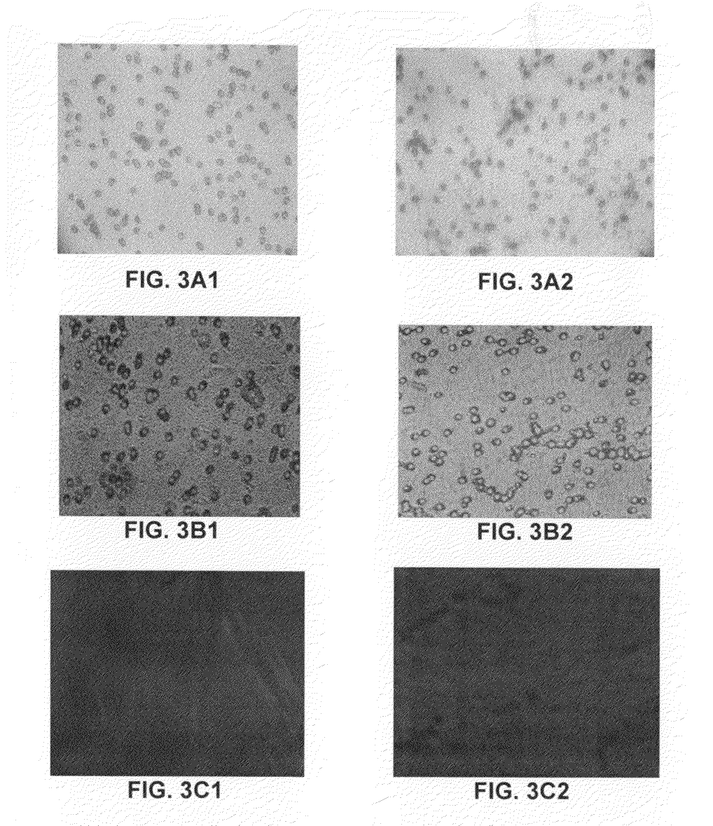

(3) Electron microscope photographs were taken, including the electron microscope photographs of FIG. 3A1, FIG. 3A2, FIG. 3B1, FIG. 3B2, FIG. 3C1 and FIG. 3C2, and records were made.

FIG. 3A1, FIG. 3A2, FIG. 3B1, FIG. 3B2, FIG. 3C1 and FIG. 3C2 show that the fibrin membrane of EXAMPLE 2 arrested dissociative tumor cells in the inserts and prevented tumor cell pervasion through the fibrin membrane and, therefore, also support the conclusion that the fibrin membrane can hold back human tumor cells and prevent pervasion. FIGS. 3A1 and 3A2 are electron microscope photographs without FS fibrin membrane showing that cell invasion occurs where there is no fibrin membrane. FIGS. 3B1 and 3B2 are the photographs of prevention of cell invasion on the inner side of the FS fibrin membrane. FIG. 3C are the photographs of prevention of cell invasion on the exterior side of the FS fibrin membrane. It is very clear that the fibrin membrane compounded by the human blood fibrinogen and thrombin does hold back human tumor cells and prevent pervasion.

It will further be appreciated by those skilled in the art and it is contemplated that variations to the embodiments illustrated and described herein may be made without departing from the spirit and scope of the present invention. Accordingly, it is intended that the foregoing description is illustrative only, and the true spirit and scope of the invention will be determined by the appended claims.

1. A kit used to compound fibrin membrane, comprising a bottle of 100-200 mg lyophilized fibrinogen with a solvent of 2 ml water for injection and a bottle of 200-2000 IU lyophilized thrombin with a solvent of 2 ml 20-60 mmol/L CaCl₂ solution.

2. The kit as claimed in claim 1, wherein the concentration of fibrinogen is 60-80 mg/ml, the concentration of thrombin is 300-800 IU/ml, and the concentration of CaCl₂ is 30-50 mmol/L CaCl₂.

3. The kit as claimed in claim 1, wherein the concentration of fibrinogen is 70 mg/ml, the concentration of thrombin is 400-600 IU/ml, and the concentration of CaCl₂ is 40 mmol/L CaCl₂.

4. The kit as claimed in any of the claims 1-3, wherein the solution of thrombin comprises CaCl₂ and thrombin.

5. The kit as claimed in any of the claims 1-3, wherein thrombin and fibrinogen are sourced from human blood.

6. The kit as claimed in any of the claims 1-3, further comprising instructions for the use of the kit.

7. The kit as claimed in any of the claims 1-3, wherein the kit is applied to produce a compounded fibrin membrane.

8. The kit as claimed in any of the claims 1-3, wherein the kit is applied in preventing dissociative tumor cell pervasion in clinical operations.

9. A method of preventing dissociative tumor cell pervasion in clinical operations, comprising:
producing a fibrin membrane on the tumor.

10. The method of claim 9, wherein the fibrin membrane is produced by applying a solution of lyophilized fibrinogen and a solution of lyophilized thrombin to the tumor.

11. The method of claim 10, wherein the solution of fibrinogen and the solution of thrombin are applied alternately to the tumor.

12. The method of claim 11, wherein the solution of fibrinogen and the solution of thrombin are each applied to the tumor 3 to 5 times.

13. The method of claim 10, wherein the solution of fibrinogen comprises 100-200 mg lyophilized fibrinogen with a solvent of 2 ml water and the solution of thrombin comprises 200-2000 IU lyophilized thrombin with a solvent of 2 ml 20-60 mmol/L CaCl₂.

14. The method of claim 10, wherein the solution of fibrinogen comprises 60-80 mg/ml, the concentration of thrombin is 300-800 IU/ml, and the concentration of CaCl₂ is 30-50 mmol/L CaCl₂.

15. The method of claim 10, wherein the solution of fibrinogen comprises 70 mg/ml, the concentration of thrombin is 400-600 IU/ml, and the concentration of CaCl₂ is 40 mmol/L CaCl₂.

16. The method of claim 10, wherein the fibrinogen and the thrombin are sourced from human blood.
